(19) United States
(12) Patent Application Publication
(10) Pub. No.: US 2014/0314538 A1
(43) Pub. Date: Oct. 23, 2014

(54) SUPPORT ARM SYSTEM FOR MEDICAL EQUIPMENT

(71) Applicant: TransEnterix Surgical, Inc., Morrisville, NC (US)

(72) Inventors: Adam Carter, Morrisville, NC (US); Keith Phillip Laby, Oakland, CA (US)

(73) Assignee: TransEnterix Surgical, Inc., Morrisville, NC (US)

(21) Appl. No.: 14/255,980

(22) Filed: Apr. 18, 2014

Related U.S. Application Data
(60) Provisional application No. 61/854,255, filed on Apr. 18, 2013.

(51) Int. Cl. A61B 19/00

(52) U.S. Cl. CPC A61B 19/26; A61B 2019/266
USPC 414/744.3

(57) ABSTRACT

A support arm for a medical device includes a column and a boom rotatably coupled to the column for rotation relative to a first axis. An arm is coupled to the boom for rotation relative to a second axis. A link mount is coupled to the arm and rotatable relative to the arm about a third axis. A device mount is supported by a linkage having link members arranged such that rotation of said first link member relative to the link mount about a fourth axis rotates the medical device about a fifth axis. The device mount is rotatable relative to the linkage about a sixth axis. The third, fifth and sixth axes extend through the center of mass of a medical instrument supported by the device mount.

### TECHNICAL FIELD OF THE INVENTION

The present invention relates to the field of support arms for supporting medical equipment such as surgical access systems or surgical instruments during a medical procedure.

### BRIEF DESCRIPTION OF THE DRAWINGS

FIG. 1A is a perspective view showing an exemplary support arm system. The arm system is shown supporting a medical device above a surgical bed.

FIG. 1B is a perspective view of the support arm system of FIG. 1A, in which the medical device is not shown.

FIGS. 2 and 3 are perspective views showing the vertical support of the support arm system of FIG. 1A.

FIG. 3A shows a lower portion of the vertical support of FIG. 3.

FIG. 4 is a perspective view of the counterbalance arm and link assembly.

FIG. 4A is a perspective view of the vertical support, counterbalance arm, and link assembly.

FIG. 5 is a perspective view of the counterbalance arm with the housing not shown.

FIG. 6 is a perspective view, with housing elements not shown, showing the components joining the counterbalance arm and link assembly.

FIG. 7 is similar to FIG. 5 but shows the components for adjusting the counterbalance force in exploded view.

FIG. 8 is a perspective view of the link assembly.

FIG. 9 is a perspective view showing an alternative embodiment of a support arm system with a medical device mounted to it.

### DETAILED DESCRIPTION

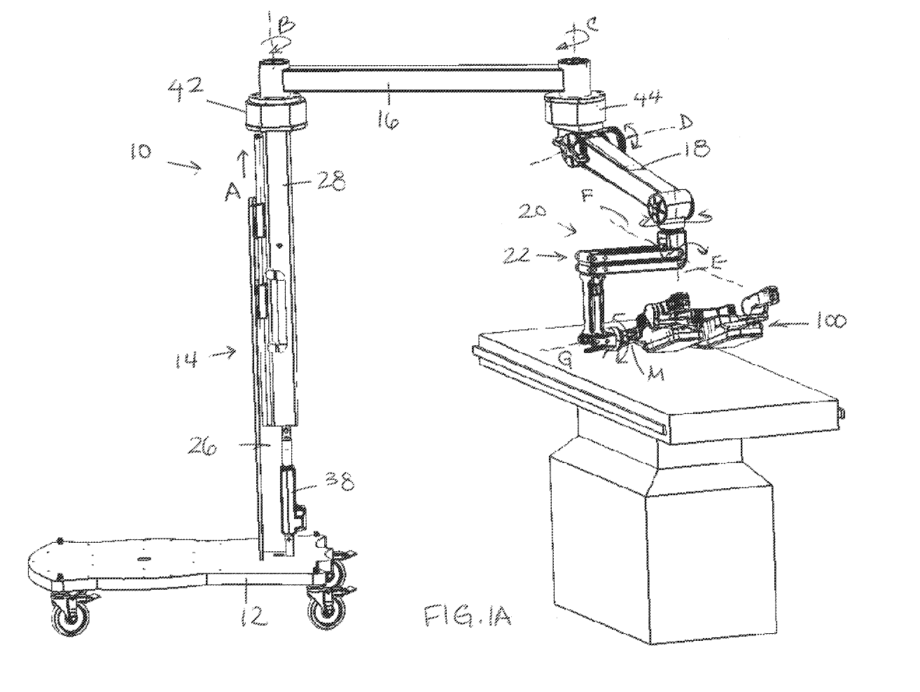

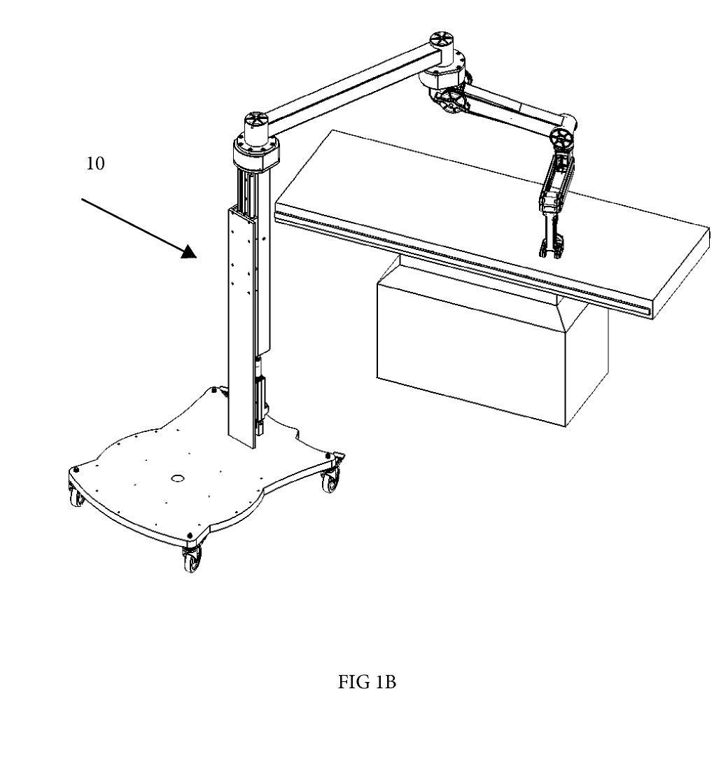

Referring to FIG. 1A, an embodiment of a support arm system 10 includes a base 12 and vertical support 14 extending from the base 12. The base is positioned on locking wheels or castors as shown.

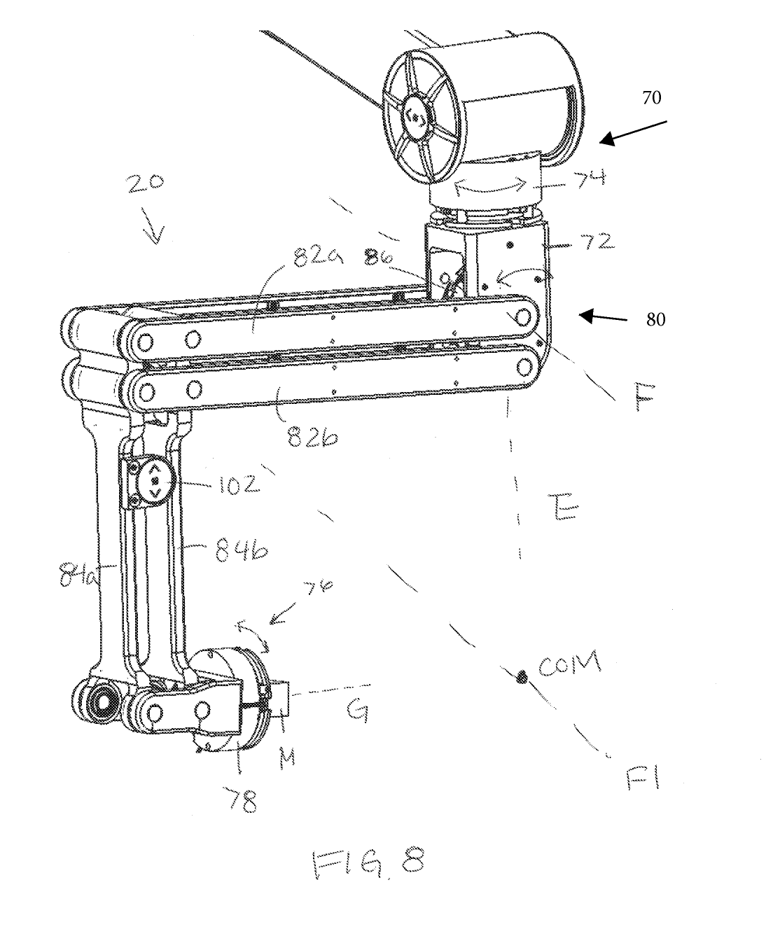

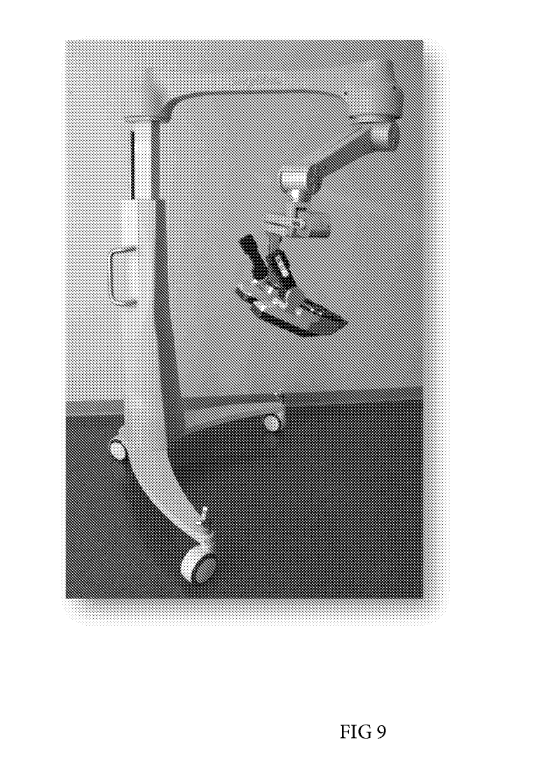

A boom 16 extends laterally from an upper portion of the vertical support 14 and carries a counterbalance arm 18. A link assembly 20 disposed on the counterbalance arm 18 includes a mount M that supports a medical device 100 for use in performing a medical procedure. The link assembly includes first parallel links 82a, 82b, (each such link shown as formed by two parallel bars) and second parallel links 84a,b (see FIG. 8) extending angularly from the first parallel links. Mount M is positioned such that the medical device 100 extends laterally from the second parallel links 84a, b. In this embodiment, the medical device is a mechanized multi-instrument surgical system of the type shown and described in U.S. Ser. No. 13/759,036, filed Feb. 4, 2013 (and incorporated herein by reference), which describes a surgical access system having steerable tubular fingers for receiving flexible instruments, multiple motors for driving the tubular fingers to steer the instruments, motors for axially rolling the instruments within the tubular fingers, user input devices that generate signals used to generate drive signals in order to drive the motors as needed to steer and roll the instruments, together with associated computer boards and related electronics. However, the support arm system may be used to support various other types of medical devices.

The system 10 is configured to allow positioning of the medical device with seven degrees of freedom. Other systems might allow for a larger or smaller number of degrees of freedom.

As illustrated in FIG. 1A, the degrees of freedom include:
elevation of the boom 16 vertically as indicated by arrow A;
rotation of the boom 16 relative to the column 28 about axis B;
rotation of the counterbalance arm 18 relative to the boom 16 about axis C;
pivoting of the distal end of the counterbalance arm 18 upwardly/downwardly relative to the proximal end, about axis D;
rotation of the link assembly 20 about axis E (which intersects the medical device's center of mass COM as shown in FIG. 8), to produce yaw motion of the medical device relative to axis E;
pivoting of link assembly 20 about axis F, which through 4-link assembly produces roll motion of the medical device 100 about remote axis of rotation F1 (which extends through the medical device's center of mass COM as shown in FIG. 8); and
rotation of the medical device 100 relative to axis G (which intersects the medical device's center of mass COM as shown in FIG. 8), corresponding to forward-aft tilt, or “pitch” of the medical device.

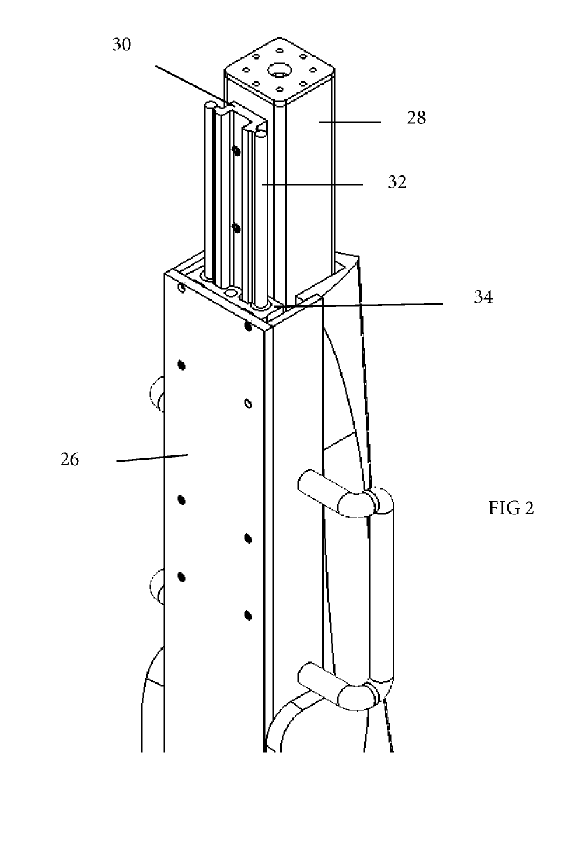

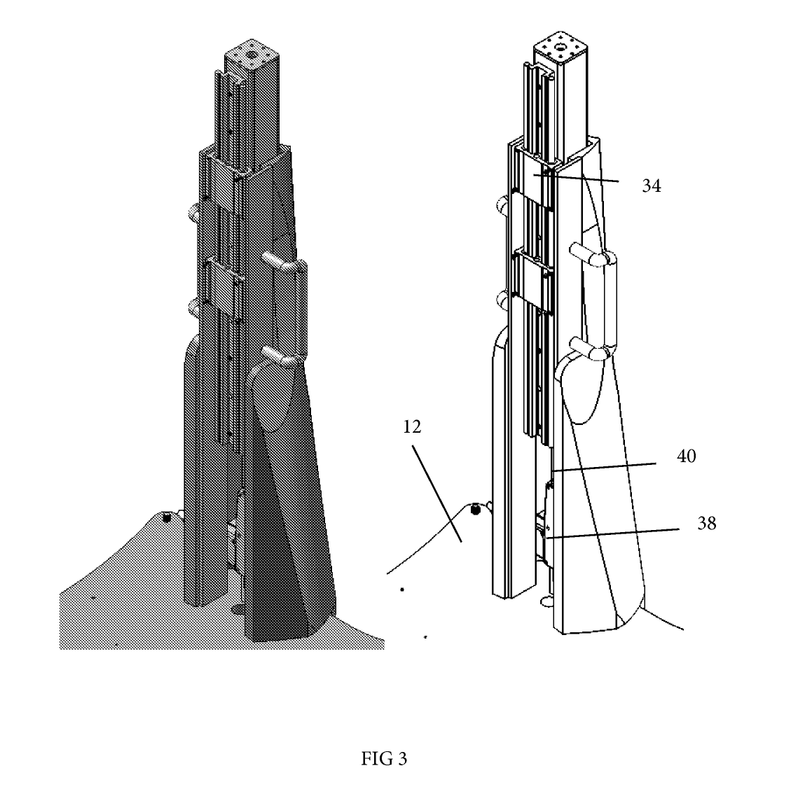

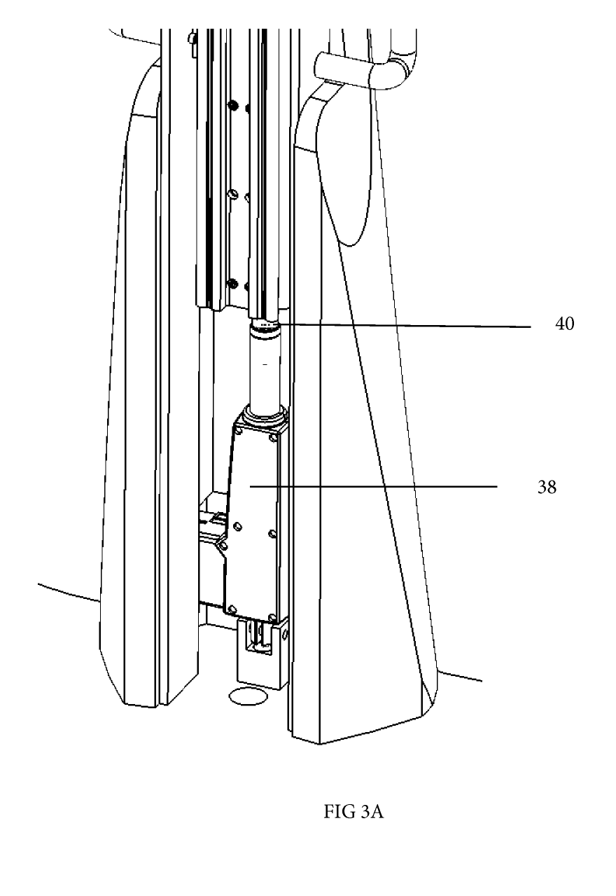

Vertical support 14 includes a lower column 26 and an upper column 28 that is vertically extendable relative to the lower column 26. As shown in FIGS. 2 and 3, a rail 30 is mounted on the upper column 28. The rail 38 includes at least one shaft 32, a pair of which are shown arranged in parallel. One or more guides 34 are mounted on the lower column 26 (two are shown in FIG. 3) and include a pair of parallel tracks 36. The parallel shafts 32 are positioned to slide within the parallel tracks 36 during upward/downward vertical movement of the upper column 28 relative to the lower column 26.

A motor 38, shown in FIGS. 3 and 3A, is mounted to the base 12 or the lower column 26. The motor includes a piston 40 coupled to the upper column 28, such that when the piston 40 extends in response to operation of the motor 38, the upper column 28 is extended relative to the lower column, and such that when the piston 40 is withdrawn, the upper column 28 is retracted towards the base 12. A user input device is operationally coupled to the motor, allowing the user to raise and lower the upper column 28 as needed. Exemplary user input devices include a switch or button, foot pedal, etc., or a voice actuated input device that responds to voice instructions.

With vertical motion having just been described, motion in the remaining six degrees of freedom will next be discussed. In general, motion in the remaining six degrees of freedom is preferably manual motion that fluidly occurs as a result of the user moving the medical device 100 towards a desired position and orientation. As will be discussed in the description that follows, a plurality of brakes are positioned such that once the user has positioned the medical device 100 in a desired position and orientation, the brakes are employed to lock the corresponding components of the arm system against each of the six degrees of freedom so as to retain the medical device 100 at the chosen position and orientation. While the application describes the use of electromagnetic breaks, other types of breaks, such as pneumatic or hydraulic brakes, might instead be used. User input for the brakes can be in the form of one or more buttons or foot pedals that activate and deactivate the brakes, or a voice control system responsive to voice commands.

In the disclosed system, the brakes are configured for simultaneous engagement, such that all may be simultaneously released to allow the user to manually position the device 100 (e.g. by manually moving the device into the desired position and orientation), and then simultaneously engaged to retain the medical device 100 in the new position/orientation. In the embodiment shown in the drawings, a switch 102 (FIG. 8) is positioned on the support arm system to allow the user to easily engage and disengage the brakes. Electronics associated with the switch are positioned on or in the components of the system, with the associated electrical cabling extending through or along the various members of the system.

In alternative embodiments, different ones or subsets of the brakes may instead be simultaneously engaged, allowing the user to alter some aspects of the medical device's position without changing others.

Referring again to FIG. 1A, brake assemblies 42, 44 (and in the illustrated the example, bearing/brake assemblies) are positioned at opposite ends of the boom 16. Bearing/brake assembly 42 is coupled between the boom 16 and the upper column 28 and, when its brake is activated, it prevents rotation of the boom 16 about axis B. Bearing/brake assembly 44 is coupled between the boom 16 and the counterbalance arm 18. When its brake is activated, it prevents rotation of the arm 18 relative to axis C.

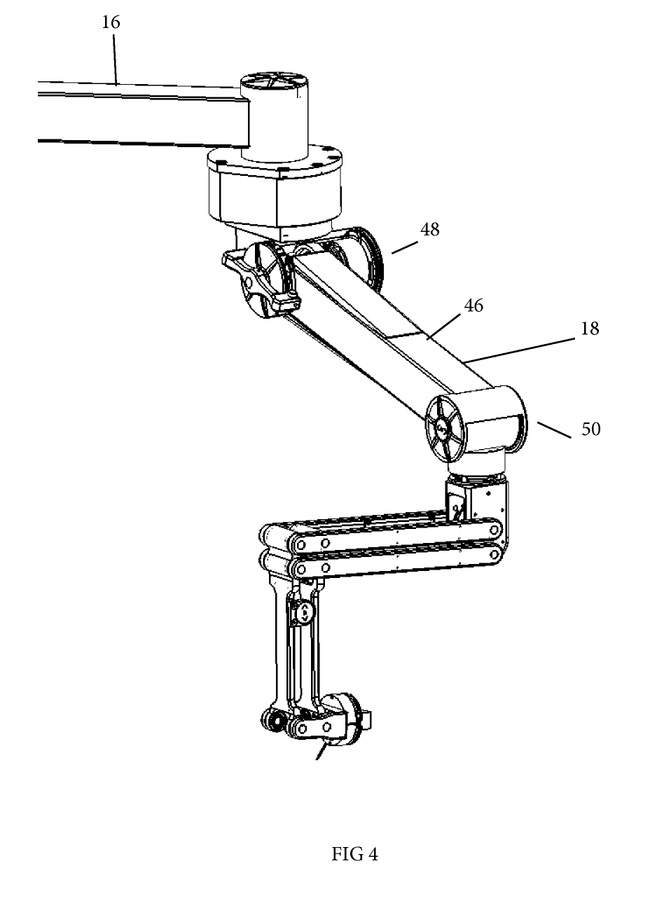

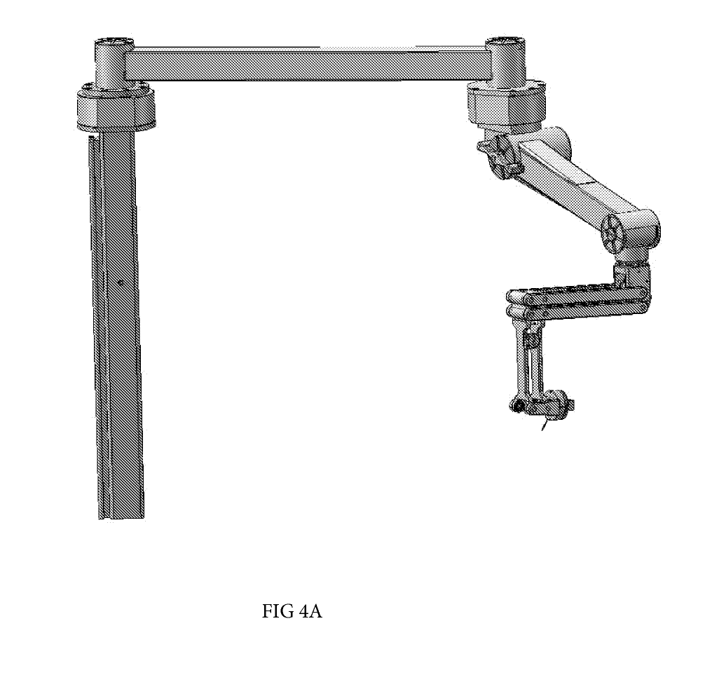

Referring to FIG. 4, counterbalance arm 18 is comprised of a tubular housing 46 that is mounted to or integrated with a hub at its proximal end for pivotal movement about axis D. The housing 46 contains counterbalance features that provide counterbalance against the mass of the medical device 100, as well as linkage features that maintain the vertical orientation of the link assembly 20 when the distal end 50 of the counterbalance arm 18 is rotated relative to its proximal end 48.

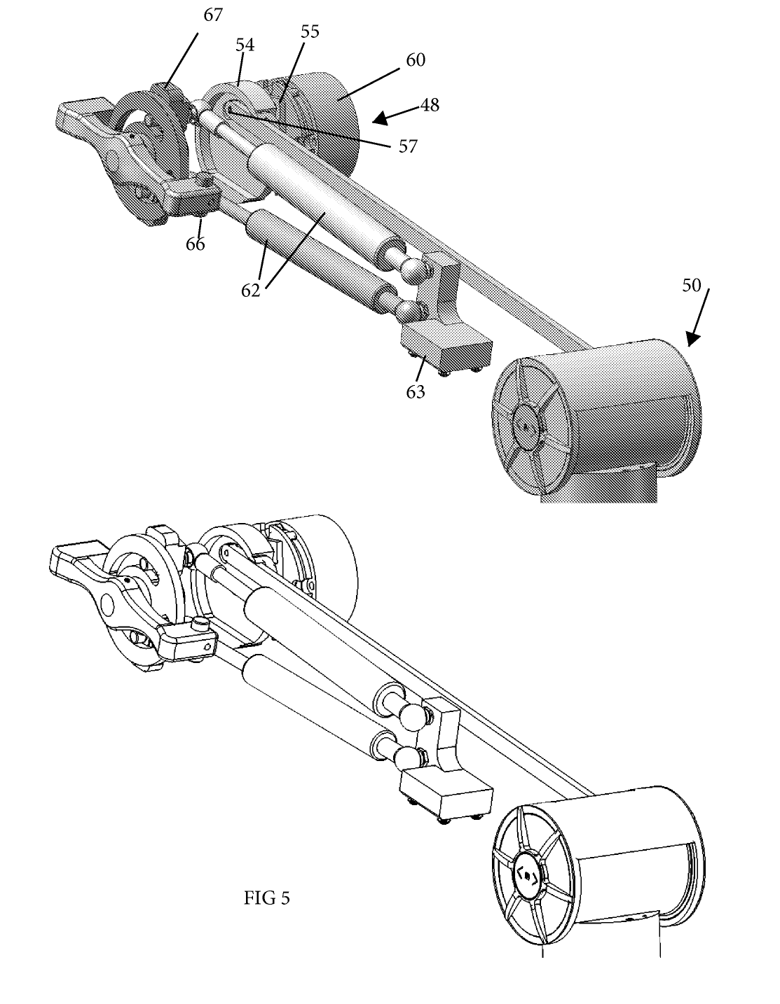

FIG. 5 shows the counterbalance arm 18 without the tubular housing 46 or the housing that contains the hub and associated components of the proximal end 48. This figures shows one component of the hub 54 to which the tubular housing 46 (FIG. 4) is mounted. Another component of the hub 54 (not shown) is positioned adjacent to slide holder 67.

A link 52 disposed within the tubular housing 46 is pivotally mounted to an internal member 55 at pivot point 57. Although the drawing shows the pivot pin for the link 52 passing through the hub 54, the internal member 55 is disposed such that it does not rotate with hub 54.

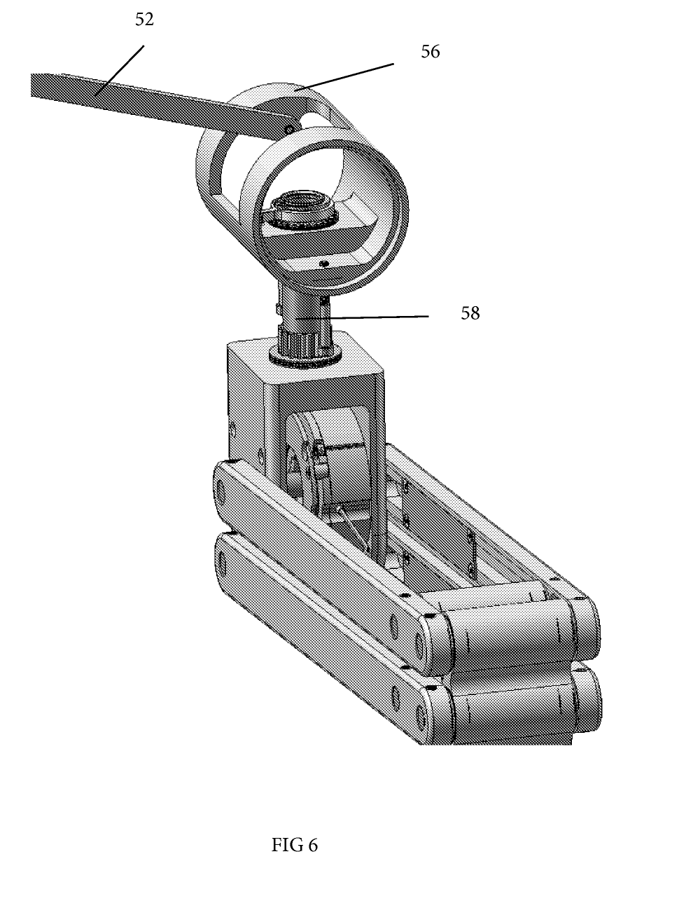

As best seen in FIG. 6, the distal end of the link 52 is pivotally coupled to a hub 56 at a location radially offset from the rotational axis of the hub 56. A vertical shaft 58 that is coupled to the link assembly 20 is mounted to the hub 56 as shown. The link 52, together with the tubular housing 46 it extends through, the hub 56, and the hub 54 form a four-bar linkage that maintains the shaft 58 in a vertical orientation regardless of the position of arm 18 relative to axis D. This keeps the planar faces of the links 82a, 82b normal to the floor as shown.

When the tubular housing 46 is pivoted about axis D, internal link 52 pivots about pivot point 57. Rotation of link 52 results in rotation of hub 56 at the link's distal end, carrying shaft such that it rotates relative to the axis of the hub 56. In this way, the vertical orientation of the shaft 58 (which in the illustrated embodiment is perpendicular to the floor) is maintained, and the link is maintained in parallel to the ground.

Referring again to FIG. 5, electromagnet brake 60 is positioned such that when it is engaged it will prevent rotation of arm 18 about axis D.

Counterbalance springs, in the form of gas springs 62 (FIG. 5), are positioned within the arm, preferable enclosed by the housing shown in FIG. 4. The distal ends of the gas springs 62 are coupled to the arm 18. In this embodiment, the distal ends are mounted to a block 63 on the tubular housing 46 (not shown in FIG. 5). The proximal ends of the gas springs are mounted to the hub 54.

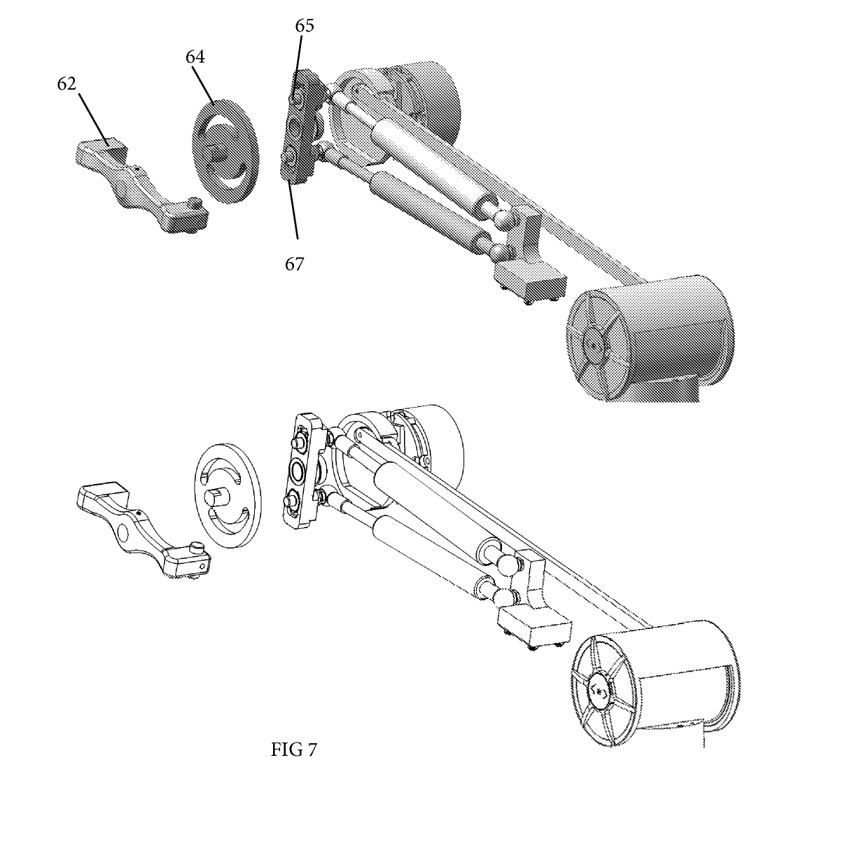

The amount of counterbalance force is optionally adjustable by means of an adjustment handle 62 as shown in the illustrated embodiment. In particular, the proximal ends of the gas springs (the pistons) are pivotally attached (via spherical pivots) to shafts 65. The shafts 65 extend through slide holder 67 and through cam slots of a cam path 64. When the adjustment handle 62 is rotated, the cam slots move the shafts 65 radially inwardly or outwardly within the slide holder 67 (FIG. 7), thus pivoting the proximal ends of the gas springs. Radial inward movement of the proximal ends of the gas springs shortens the effective lever arms provided by the gas springs and thus produces a lower counterbalance force. Radial outward movement of the gas springs lengthens the effective lever arms provided by the gas springs, and thus increases the counterbalance force. Additional gas springs may be added if additional counterbalance forces are required. For example, a third gas spring may extend between the block 63 and the hub 54.

A latch is provided for engaging the position of the adjustment handle 62 relative to the surrounding housing (housing shown in FIG. 4) so as to fix the orientation of the gas springs and to thereby set the desired amount of counterbalance force. In the illustrated embodiment, a pin extends from the adjustment handle 62 into engagement with a select one of a plurality of circumferential openings on the housing surrounding the hub. Depressing a button 66 against a spring retracts the pin from the opening, enabling the user to rotate the adjustment handle 62 to adjust the counterbalance force. After the adjustment handle has been rotated to a new position, the button 66 is released, causing the pin to radially advance into another of the circumferential openings on the housing, thus retaining the proximal ends of the gas spring positions in their new positions and thereby setting the counterbalance force.

Pitch, roll and yaw positioning of the medical device (not shown) is accomplished using the features shown in FIG. 8. As discussed, the associated joints and links are configured such that the axis of rotation for each of the pitch, roll and jaw motions occurs at the center of mass (COM) of the medical device. In particular, a rotation joint 70 allows for rotation of a link mount 72 about axis E (which intersects the COM of the medical device) for yaw motion of the medical device 100. Electromagnetic brake 74, when engaged, prevents such motion.

Similarly, rotation joint 76 at mount M allows for rotation of medical device 100 (see FIG. 1A) relative to the link assembly 20, about axis G for pitch (or forward-aft tilt) of the medical device. Axis G intersects the COM of the medical device. Brake 78 is positioned to prevent such motion when it is engaged.

Finally, roll movement of the medical device 100 relative to roll axis F1 is achieved by way of rotation joint 80 in combination with movement of the four link assembly 20. Upper link 82a rotates about axis F, and the resulting movement of the four link assembly results in rotation of the medical device 100 about axis F1, which intersects the COM and which is perpendicular to the axes G and E. When brake 86 is engaged, it restricts rotation at rotation joint 80.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed. Moreover, features of the various disclosed embodiments may be combined in various ways to produce various additional embodiments.

Any and all patents, patent applications and printed publications referred to above, including for purposes of priority, are incorporated herein by reference.

We claim:

1. A support arm for supporting a medical device having a center of mass, the support arm comprising:
a support;
a link mount coupled to the support and rotatable relative to the support about a first axis;
a linkage comprising a plurality of link members and a device mount carried by the link members, the device mount releasably couplable to a medical device, a first one of the link members rotationally coupled to the link mount, the link members arranged such that rotation of said first link member relative to the link mount about a second axis rotates the medical device about a third axis;
the device mount rotatable relative to the linkage about a fourth axis;
wherein the first, third and fourth axes extend through the center of mass of the medical instrument.

2. The system of claim 1, wherein the first, third, and forth axes intersect at the center of mass.

3. The system of claim 2, wherein the second and third axes are parallel to one another.

4. The system of claim 1, further including a plurality of brakes engageable to prevent rotation about the first, second, and third axes.

5. The system of claim 4, further including a switch for simultaneously engaging each of the brakes in the plurality of brakes.

6. A support arm for a medical device, comprising:
a column;
an arm coupled to the column, the arm including a four bar linkage comprised of a first hub coupled to the column at a first end of the arm, a second hub at a second end of the arm, a tubular arm member, and a link extending through the tubular arm member; and
a mount coupled to the second end of the arm for supporting a medical device.

7. The support arm of claim 7, further including a plurality of gas springs disposed within the tubular arm member, each gas spring having a first end coupled to the first hub, and a second end coupled to the tubular arm member.

8. The support arm of claim 6, wherein the mount includes a link mount and linkages of the type recited in claim 1.

9. A support arm for a medical device, comprising:
a base;
a vertically extendable column positioned on the base;
a boom rotatably coupled to the column for rotation relative to a first axis, and a first brake positioned to prevent rotation of the boom relative to the column;
an arm rotatably coupled to the boom for rotation relative to a second axis, and a second brake positioned to prevent rotation of the arm relative to the boom;
a link mount coupled to the arm and rotatable relative to the arm about a third axis and a third brake positioned to prevent rotation of the link mount about the third axis;
a linkage comprising a plurality of link members and a device mount carried by the link members, the device mount releasably couplable to a medical device, a first one of the link members rotationally coupled to the link mount, the link members arranged such that rotation of said first link member relative to the link mount about a fourth axis rotates the medical device about a fifth axis;
the device mount rotatable relative to the linkage about a sixth axis;
a plurality of brakes engagable to prevent rotation about the third, fourth, and sixth axes, respectively;
wherein the third, fifth and sixth axes extend through the center of mass of the medical instrument.

10. The system of claim 9, further including a switch for simultaneously engaging the brakes.
